(19) United States
(12) Patent Application Publication
(10) Pub. No.: US 2012/0129966 A1
(43) Pub. Date: May 24, 2012

(54) PROCESS FOR PRODUCING POLYESTER POLYOLS WITH LOW QUANTITIES OF DIOXANE WASTE

(75) Inventors: Hartmut Nefzger, Pulheim (DE); Erika Bauer, Juchen (DE); Johannes Van De Braak, Hennef (DE); Silvia Kasperek, Wesseling (DE)

(73) Assignee: Bayer MaterialScience AG, Leverkusen (DE)

(21) Appl. No.: 13/376,450

(22) PCT Filed: Jun. 2, 2010
(86) PCT No.: PCT/EP2010/003359

§ 371 Date: Jan. 23, 2012

(30) Foreign Application Priority Data
Jun. 10, 2009 (DE) 102009024479.4

(51) Int. Cl. C08G 18/42; C07C 69/82; C07C 67/08

(52) U.S. Cl. 521/159; 560/91

(57) ABSTRACT

The present invention relates to the production and use of polyester polyols produced from at least one aromatic dicarboxylic acid or an alkyl ester of an aromatic dicarboxylic acid or the anhydride of an aromatic dicarboxylic acid and at least one α,ω-diol, wherein the formation of 1,4-dioxane from diethylene glycol is largely suppressed by means of specific reaction management.

### BACKGROUND OF THIS INVENTION

The present invention relates to the production and use of polyester polyols built up from at least one α,ω-diol and one aromatic dicarboxylic acid or mixtures of these or the alkyl esters thereof or the anhydrides thereof and diethylene glycol, wherein the formation of 1,4-dioxane from diethylene glycol is largely suppressed by means of specific reaction control.

Polyester polyols are an important constituent of many foamed and non-foamed polyurethane systems. Polyester polyols as used for the formation of polyurethanes have predominantly hydroxyl end groups, which are available for further reaction with isocyanate groups. The molecular weight of the polyester polyols is typically in the range of 200-5000 daltons. Their production takes place mainly by polycondensation of polycarboxylic acids, in particular dicarboxylic acids, and polyols, in particular diols, by reacting carboxyl and hydroxyl groups under dewatering conditions with the formation of ester groups. Alternatively, it is also possible to use anhydrides of polycarboxylic acids, e.g. phthalic anhydride, or internal anhydrides, i.e. lactones.

Dewatering conditions can be achieved for example by applying a vacuum, blowing out the water of reaction using an inert gas stream or azeotropic distillation using an entrainer (Houben-Weyl, Methoden der organischen Chemie, vol. 14/2, Makromolekulare Stoffe, Thieme Verlag Stuttgart, ed. E. Müller, pp. 1-47, 1963).

It is known to the person skilled in the art that, during esterification of aromatic polycarboxylic acids, such as e.g. terephthalic, isophthalic or phthalic acid, usually used in the form of phthalic anhydride, with diethylene glycol 1,4-dioxane is undesirably formed as a by-product. The dioxane formed during production in industrial plants is discharged together with the water of reaction and must then, for example, be broken down in water treatment plants or incinerated after concentration. This additional process step leads to an increase in the costs of polyester polyol production.

The 1,4-dioxane formed as a by-product also leads to a reduction in the yield of desired product, since part of the diethylene glycol used is not incorporated into the polyester being produced but is removed from the reaction mixture in the form of 1,4-dioxane, as described. Thus, the formation of 1,4-dioxane creates a serious economic disadvantage.

Furthermore, the quantity of 1,4-dioxane that a production plant is allowed to produce may be restricted by licensing parameters. In these cases, therefore, restriction of the quantity of dioxane leads indirectly to a restriction of the production capacity of a plant for producing polyester polyols.

### SUMMARY OF THE INVENTION

It is therefore an object of the present invention to provide a process for producing polyester polyols built up from at least one α,ω-diol and terephthalic and/or isophthalic acid and diethylene glycol, which overcomes the disadvantages of the prior art.

In particular, it is an object of the present invention to restrict the quantity of dioxane formed in relation to the quantity of diethylene glycol used during the production of polyester polyols from at least one α,ω-diol and terephthalic and/or isophthalic acid and diethylene glycol. The quantity of dioxane here can be restricted to less than 20 g per kg, preferably to less than 10 g per kg of diethylene glycol used.

It is a further object of the present invention to reduce the quantity of dioxane formed in relation to the quantity of polyester polyol formed during the production of polyester polyols from at least one α,ω-diol and terephthalic and/or isophthalic acid and dicthylene glycol. The quantity of dioxane here can be restricted to less than 4 g per kg, preferably to less than 3 g per kg of polyester polyol formed.

The object mentioned above is achieved by the process according to the invention.

The invention relates to a process for producing polyester polyols wherein, in one step,
c) at least one aromatic dicarboxylic acid or alkyl ester of an aromatic dicarboxylic acid or anhydride of an aromatic dicarboxylic acid (A) and at least one α,ω-diol (B) are mixed,
wherein the molar ratio of components (B):(A) is in the range of 20:1 to 1.05:1 and the proportion by weight of components (A) and (B) in total, based on the weight of the mixture obtained in step c), is in the range of between 55 and 99 wt. %, with the formation of a polyester polyol, and to the polyester polyol obtained in step a), phthalic anhydride (C) is added in the molar ratio of components (C) to the at least one aromatic dicarboxylic acid or alkyl ester of the aromatic dicarboxylic acid or anhydride of an aromatic dicarboxylic acid (A) added in step a) in the range of 0:1 to 2:1, preferably 0.1:1 to 1.5:1, with the formation of a polyester polyol, and
c) to the polyester polyol obtained in step b), diethylene glycol (D) is added in a proportion by weight of 1 to 35 wt. %, based on the weight of the mixture, with the formation of a polyester polyol,
wherein the polyester polyol obtained in step b) has a higher molecular weight than the polyester polyol obtained in step c).

Step b) can, of course, be omitted in the event that the molar ratio of components (C) to the at least one aromatic dicarboxylic acid (A) added in step a) assumes a value of 0.

The components lacking in step a) in an amount of 100 wt. %, based on the weight of the mixture obtained in step c), can be added in steps h) and/or c), but can also be added as an additional component (E) in addition to components (A), (B), (C) or (D) in steps a) and/or b) and/or c). The mixture obtained in step c), which contains the components added in steps a)-c) or the reaction products thereof, is thus the basis of the weight data. These additional components (E) are preferably low-molecular-weight polyols, for example 1,1,1-trimethylolpropane, pentaerythritol or glycerol, or branched polyethers and/or polyester polyols with number-average functionalities greater than 2. These additional components (E) are preferably added in step a).

### DETAILED DESCRIPTION OF THE INVENTION

The carboxylic acids (A) are aromatic and are at least one of the isomeric phthalic acids, i.e. phthalic acid, isophthalic acid or terephthalic acid or mixtures thereof. Instead of the aromatic carboxylic acids or in a mixture with the aromatic carboxylic acids, it is also possible to use the alkyl esters or anhydrides thereof, in particular phthalic anhydride. The alkyl esters are preferably diesters, in which both carboxyl groups are esterified. In this case, the two ester groups can be the same or different. Alkyl here means preferably C₁-C₂₀ alkyl, particularly preferably methyl, ethyl, propyl, n-butyl, iso-butyl, pentyl with the isomers thereof, hexyl with the isomers thereof, heptyl with the isomers thereof, octyl with the isomers thereof; nonyl with the isomers thereof, decyl with the isomers thereof and C₁₁, C₁₂, C₁₃, C₁₄, C₁₅, C₁₆, C₁₇, C₁₈, C₁₉, C₂₀, with the isomers thereof in each case.

The aromatic dicarboxylic acid (A) is preferably selected from the group consisting of terephthalic acid and isophthalic acid: most particularly preferably the aromatic dicarboxylic acid is terephthalic acid, including the dialkyl esters thereof, in which case instead of water, alkyl alcohol is formed as a condensation product.

As α,ω-diols (B), all di-primary diols except diethylene glycol are suitable.

Preferred α,ω-diols (B) are, for example, ethylene glycol, triethylene glycol, tetraethylene glycol, pentaethylene glycol and the higher homologs thereof with number-average molecular weights of 150 to 410 g/mol.

According to the invention, the addition of the diethylene glycol (D) in step c) and equilibration to the polyester equilibrium take place such that the distribution of the individual oligomers of the polyester polyol corresponds to Flory's oligomer distribution function (P. J. Flory, Principles of Polymer Chemistry, Cornell University Press, Ithaca 1953, page 317 ff.). Polyester polyols of a specified type in Flory's equilibrium always have the same oligomer distribution and thus give consistent material properties in respect of the polyurethane materials made thereof.

The addition of the diethylene glycol (D) takes place in step c) at any temperature, both of the polyester polyol obtained from step b), or optionally from step a), and of the diethylene glycol to be added. The diethylenc glycol to be added subsequently preferably has a temperature from room temperature (20° C.) to 60° C., and the polyester polyol from step b), or optionally from step a), an elevated temperature from 120 to 200° C. The diethylene glycol (D) is added under laboratory conditions in a nitrogen counter-current, and under industrial conditions is preferably sucked into the vessel by applying a vacuum. When equal amounts of the same feedstocks are used, the properties of the polyester polyols produced according to the invention differ from polyester polyols not produced according to the invention not only in terms of the yields but also in terms of their properties. Under these conditions, for example, the hydroxyl number of polyester polyols according to the invention is greater or their molecular weight smaller than that of polyester polyols not produced by the process according to the invention. The same also applies to polyurethane (PUR) produced therefrom. This is the result of the dioxane formed from diethylene glycol in the case that is not according to the invention.

In the case that is not according to the invention, of course, the partial loss of diethylene glycol can be compensated by compensating for the amount of diethylene glycol lost as dioxane in advance or by subsequent addition. In this case, similar polyester polyols are obtained; however, the formulations then differ in that the amount of feedstocks, in particular of diethylene glycol, then needed to synthesise a given amount of polyester polyol with the same gross composition is disadvantageously increased in the process that is not according to the invention.

The amount of diethylene glycol (D) to be added is determined by the OH number of the product from step b), or optionally from step a), and the OH number of the desired end product and from the batch size, according to the following formula:

added diethylene glycol (D) in g=(Z−Y)*M/(1058−Z)

with

target OH number after step c) Z

OH number found from step b), or optionally from step a), Y

amount of polyester from step b), or optionally from step a), in g M

OH number of diethylene glycol 1058

The subsequent addition of diethylene glycol (D) can take place distributed over a prolonged period, e.g. over 1 to 5 hours, continuously, uniformly or non-uniformly or in one shot.

The molar ratio of components (B):(A) is preferably in the range of 1.1:1.0 to 10.0:1.0.

The OH number of the polyester polyol obtained from step a) is preferably in the range of between 28 and 560 KOH/kg, preferably in the range of between 37 and 450 KOH/kg.

The molecular weight of the polyester polyol obtained from step a) is preferably in the range of between 4000 and 200, preferably in the range of between 3000 and 250 g/mol.

The OH number of the polyester polyol obtained from step b) is preferably in the range of between 25 and 190 g KOH/kg, preferably in the range of between 28 and 160 g KOH/kg.

The molecular weight of the polyester polyol obtained from step b) is preferably in the range of between 4500 and 600, preferably in the range of between 4000 and 700 g/mol.

The viscosity of the polyester polyol obtained from step b) at a temperature of 25° C. is preferably in the range of between 400 and 20000 mPas, preferably in the range of between 450 and 15000 mPas.

The proportion by weight of components (A) and (B) in step a) in total, based on the weight of the mixture, is preferably in the range of between 60 and 90 wt. %.

In step b), phthalic anhydride (C) is preferably added to the polyester polyol obtained in step a) in the molar ratio of components (C) to the at least one aromatic dicarboxylic acid (A) added in step a) in the range of 0:1 to 1.5:1.

The OH number is determined by reacting the hydroxyl end groups in a sample of the polyester polyol firstly with a defined excess of an anhydride, e.g. acetic anhydride, hydrolysing the excess anhydride and determining the content of free carboxyl groups by direct titration with a strong base, e.g. sodium hydroxide. The difference between the carboxyl groups introduced in the form of the anhydride and the experimentally determined carboxyl groups is a measure of the number of hydroxyl groups in the sample. Provided that this value is corrected by the number of carboxyl groups contained in the original sample as a result of incomplete esterification, i.e. by the acid number, the OH number is obtained. The titrations, generally performed with sodium hydroxide, are converted to the equivalent amount of potassium hydroxide so that acid and hydroxyl numbers are expressed in g KOH/kg. The following arithmetical connection exists between hydroxyl number (OH #) and number-average molecular weight (M): M=(56100*F)/OH #. F here means the number-average functionality and can be derived from the formulation in a good approximation.

The viscosity is determined using a cone-plate viscometer, e.g. Physica MCR 51 from Anton Paar, extrapolating to a zero shear rate. Polyols according to the invention are, for the most part, not intrinsically viscous.

The polyester polyols obtained from step c) preferably have acid numbers in the range of 0.5 to 3.5.

A vacuum process is preferably carried out to produce the polyester polyols according to the invention at absolute pressures in the range of 1.1 bar to 5 mbar, particularly preferably 1.0 bar to 10 mbar, and temperatures in the range of 100-250, preferably 180 to 240° C.

The process for producing the polyester polyols according to the invention is preferably carried out by initially charging the components (B) from step a), adding the components (A) while stirring and firstly and preferably at absolute pressures of 1.0 to 1.1 bar, using a protective gas, at temperatures in the range of 100 to 240° C., particularly preferably at temperatures in the range of 180 to 240° C., condensing until phthalic acid, phthalic anhydride, terephthalic acid and/or isophthalic acid and/or the alkyl esters thereof have dissolved to form a clear solution. Then, optionally after adding an esterification catalyst, the absolute pressure is preferably reduced over the course of 1 to 4 hours to less than 100 mbar and finally, at temperatures in the range of 180 to 240° C. and absolute pressures in the range of preferably 10 to 100 mbar, polycondensation is carried out until an acid number of less than 5 g KOH/kg is obtained.

To produce the polyester polyols according to the invention, any catalysts known to the person skilled in the art can be used. Tin(II) chloride and titanium tetra-alkoxylates are preferably used.

The reaction of components to produce the polyester polyols according to the invention preferably takes place without solvents.

Alternatively, the polyester polyols can also be produced by the nitrogen blowing process, in which the condensate is driven out of the reaction vessel by a nitrogen stream (J. H. Saunders and H. T. Frisch in Polyurethanes: Chemistry and Technology, Part I. Chemistry, Interscicnce published by John Wiley and Sons, New York 1962, page 45).

The invention also relates to the polyester polyols obtainable by the process according to the invention.

The polyester polyols produced by the process according to the invention arc preferably used for producing polyurethanes. Polyurethane is a versatile material which is used in many areas. Owing to the wide variety of raw materials that can be used, products with extremely varied properties can be produced, for example rigid foams for insulation, flexible block foams for mattresses, moulded flexible foams for car seats and seat cushions, acoustic foams for sound insulation, thermoplastic foams, shoe foams or microcellular foams, but also compact casting systems and thermoplastic polyurethanes.

The present invention also provides a process for producing a polyurethane foam which may optionally contain polyisocyanurate groups (PUR-PIR foam), in which
a) polyester polyol is produced by the process according to the invention and the polyester polyol thus obtained is then reacted
b) with at least one polyisocyanate-containing component,
c) in the presence of one or more blowing agents,
d) in the presence of one or more catalysts and
e) optionally in the presence of one or more flame retardants and optionally in the presence of additional auxiliary substances and additives and
f) optionally with at least one compound having at least two isocyanate-reactive groups, which differs from the polyester polyol from step a).

The invention also relates to the PUR-PIR foams obtainable by the process according to the invention.

As the polyisocyanate-containing component, the conventional aliphatic, cycloaliphatic and in particular aromatic di- and/or polyisocyanates are suitable. Toluene diisocyanate (TDI), diphenylmethane diisocyanate (MDI) and in particular mixtures of diphenylmethane diisocyanate and polyphenylene polymethylene polyisocyanates (polymeric MDI) are preferably used. The isocyanates can also be modified, for example by incorporating uretdione, carbamate, isocyanurate, carbodiimide, allophanate and in particular urethane groups. For the production of rigid polyurethane foams, in particular polymeric MDI is used. Isocyanurate formation takes place in the prior art virtually exclusively during the foaming reaction and leads to flame-resistant PUR/PIR foams, which are preferably used in industrial rigid foam, for example in the construction sector as insulation boards, sandwich elements, pipe insulation and lorry bodies.

As compounds with at least two isocyanate-reactive groups, i.e. with at least two hydrogen atoms that are reactive with isocyanate groups, it is generally possible to use compounds which are described in general terms below.

As compounds with at least two isocyanate-reactive groups, in particular those carrying two or more reactive groups selected from OH groups, SH groups, NH groups, NH₂ groups and CH-acidic groups, such as e.g. 3-diketo groups, in the molecule are suitable. To produce the rigid polyurethane foams preferably produced by the process according to the invention, in particular compounds with 2 to 8 OH groups are used. Polyether polyols and/or polyester polyols are preferably used. The hydroxyl number of the polyether polyols and/or polyester polyols used is preferably 25 to 850 mg KOH/g, particularly preferably 25 to 550 mg KOH/g, in the production of rigid polyurethane foams, and the molecular weights are preferably greater than 300 g/mol. The component (f) preferably contains polyether polyols, which are produced by known processes, for example by anionic polymerisation with alkali hydroxides, such as sodium or potassium hydroxide, or alkali alcoholates, such as sodium methylate, sodium or potassium ethylate or potassium isopropylate as catalysts and with the addition of at least one starter molecule containing 2 to 8, preferably 2 to 6, bound reactive hydrogen atoms, or by cationic polymerisation with Lewis acids, such as antimony pentachloride, borofluoride etherate etc. or bleaching clay as catalysts, from one or more alkylene oxides with 2 to 4 carbon atoms in the alkylene residue. Furthermore, the production of the polyether polyols can take place by double metal cyanide catalysis, in which case continuous operation is also possible.

Suitable alkylene oxides are e.g. tetrahydrofuran, 1,3-propylene oxide, 1,2- and 2,3-butylene oxide, styrene oxide and preferably ethylene oxide and 1,2-propylene oxide. The alkylene oxides can be used individually, alternately in succession or as mixtures. Suitable starter molecules include, for example, glycerol, trimethylol-propane, pentaerythritol, sucrose, sorbitol, methylamine, ethylamine, isopropylamine, butylamine, benzylamine; aniline, toluidine, toluenediamine, naphthylamine, ethylenediamine, diethylenetriamine, 4,4′-methylene dianiline, 1,3-propanediamine, 1,6-hexanediamine, ethanolamine, diethanolamine, triethanolamine and other dihydric or polyhydric alcohols, which in turn can also be oligoether polyols or mono- or polyvalent amines, and water.

In addition, the component (f) may optionally contain polyester polyols, chain extenders and/or crosslinking agents. As chain extenders and/or crosslinking agents, in particular bifunctional or trifunctional amines and alcohols, in particular diols and/or triols with molecular weights of less than 400 g/mol, preferably of 60 to 300, are used. As compound (f), preferably polyether polyols and/or polyester polyols with a hydroxyl number greater than 160, particularly preferably greater than 200 mg KOH/g and particularly preferably with a functionality of between 2.9 and 8 are used. Particularly preferably, polyether polyols having an equivalent weight, i.e. molecular weight divided by the functionality, of less than 400 g/mol, preferably less than 200 g/mol, are used as isocyanate-reactive compounds (f). Compound (f) is generally present in liquid form.

As blowing agent component (c), hydrocarbons are preferably used. These can he used in a mixture with water and/or additional physical blowing agents. These are understood to mean compounds which are dissolved or emulsified in the feedstocks for polyurethane production and evaporate under the conditions of polyurethane formation. They are, for example, hydrocarbons, halogenated hydrocarbons and other compounds, such as e.g. perfluorinated alkanes, such as perfluorohexane, chlorofluorocarbons, and ethers, esters, ketones and/or acetals.

The blowing agent component (c) is used preferably in a quantity of 2 to 45 wt. %, preferably 3 to 30 wt. %, particularly preferably 4 to 20 wt. %, based on the total weight of components (b) to (f). In one preferred embodiment, the blowing agent mixture (c) contains hydrocarbons, in particular n-pentane and/or cyclopentane, and water. Particularly preferred hydrocarbons arc n-pentane, cyclopentane, iso-pentane and/or mixtures of the isomers. In particular, cyclopentane and/or n-pentane are used as blowing agents (c).

As catalysts (d) for the production of the polyurethane and polyisocyanurate foams according to the invention, the conventional and known polyurethane- or polyisocyanurate-forming catalysts are used, for example organic tin compounds, such as tin diacetate, tin dioctoate, dibutyltin dilaurate, and/or strongly basic amines, such as 2,2,2-diazabicyclooctane, triethylamine or preferably triethylenediamine, N,N-dimethylcyclohexylamine or bis(N,N-dimethylaminoethyl) ether, and to catalyse the PIR reaction potassium acetate, potassium octoate and aliphatic quaternary ammonium salts.

The catalysts are preferably used in a quantity of 0.05 to 3 wt. %, preferably 0.06 to 2 wt. %, based on the total weight of all the components.

The reaction of the above-mentioned components optionally takes place in the presence of (e) additives, such as e.g. flame retardants, fillers, cell regulators, foam stabilisers, surface-active compounds and/or stabilisers against oxidative, thermal or microbial degradation or ageing, preferably flame retardants and/or foam stabilisers. Substances which promote the formation of a regular cell structure during foam formation are referred to as foam stabilisers. The following are mentioned as examples: silicone-containing foam stabilisers, such as siloxane-oxyalkylene copolymers and other organopolysiloxanes, and also alkoxylation products of fatty alcohols, oxo alcohols, fatty amines, alkyl phenols, dialkyl phenols, alkyl cresols, alkyl resorcinol, naphthol, alkyl naphthol, naphthylamine, aniline, alkyl aniline, toluidine, bisphenol A, alkylated bisphenol A, polyvinyl alcohol, and in addition, alkoxylation products of condensation products of formaldehyde and alkyl phenols, formaldehyde and dialkyl phenols, formaldehyde and alkyl cresols, formaldehyde and alkyl resorcinol, formaldehyde and aniline, formaldehyde and toluidine, formaldehyde and naphthol, formaldehyde and alkyl naphthol, and formaldehyde and bisphenol A. As alkoxylation reagents it is possible to use e.g. ethylene oxide, propylene oxide, poly-THF and higher homologs.

As flame retardants, in general the flame retardants known from the prior art can be used. Suitable flame retardants are e.g. brominated ethers (e.g. Ixol® B251), brominated alcohols, such as dibromoneopentyl alcohol, tribromoneopentyl alcohol and PHT-4-diol, and chlorinated phosphates, such as e.g. tris(2-chloroethyl)phosphate, tris(2-chloroisopropyl)phosphate (TCPP), tris(1,3-dichloroisopropyl)phosphate, tris(2,3-dibromopropyl)phosphate and tetrakis(2-chloroethyl)ethylene diphosphate. As well as the already mentioned halogen-substituted phosphates, inorganic flame retardants, such as red phosphorus, preparations containing red phosphorus, aluminium oxide hydrate, antimony trioxide, ammonium polyphosphate and calcium sulfate, or cyanuric acid derivatives, such as e.g. melamine, or mixtures of at least two flame retardants, such as e.g. ammonium polyphosphates and melamine, and optionally starch can be used to flameproof the rigid PUR or PUR/PIR foams according to the invention. As additional liquid halogen-free flame retardants it is possible to use diethyl ethane phosphonate (DEEP), triethyl phosphate (TEP), dimethyl propyl phosphonate (DMPP), diphenyl cresyl phosphate (DPK) and others. The flame retardants are used within the framework of the present invention preferably in a quantity of 0 to 30 wt. %, particularly preferably 2 to 25 wt. %, in particular 2.5 to 15 wt. %, based on the total weight of components (b) to (e).

Further details of the above-mentioned and other starting materials can be taken from the specialist literature, for example the Kunststoffhandbuch, vol. VH, Polyurethane, Carl Hanser Verlag Munich, Vienna, 1st, 2nd and 3rd editions 1966, 1983 and 1993.

To produce the rigid polyurethane foams, the polyisocyanates (b) and the components (a) and optionally (f) are reacted in quantities such that the isocyanate index of the foam is 90 to 600, preferably 150 to 500, particularly preferably 180 to 450.

The rigid polyurethane foams can be produced batchwise or continuously using known processes. Those known to the person skilled in the art include, inter alia, block foam production (continuous and batchwisc), use in one-component systems (batchwise) and in moulded insulating foam (batchwise). The invention described here relates to all processes, but preferably to the continuous double belt process, wherein flexible and/or rigid materials can be used as covering layers.

The rigid polyurethane foams according to the invention preferably have a closed cell ratio greater than 90%, particularly preferably greater than 95%.

The PUR and PUR/PIR foams according to the invention preferably have a density of 28 g/m³ to 300 g/m³, particularly preferably 30 g/m³ to 50 g/m³.

The rigid polyurethane foams according to the invention are used in particular for thermal insulation, for example in cooling equipment, containers or buildings, e.g. in the form of insulated pipes, sandwich elements, insulation boards or cooling equipment.

Polyurethanes within the meaning of the present patent application arc also understood to include polymeric isocyanate adducts, which also contain other groups in addition to urethane groups, as formed for example by reaction of the isocyanate group with itself, for example isocyanuratc groups, or which are formed by reaction of the isocyanate groups with groups other than hydroxyl groups, said groups generally being present in the polymer together with the urethane groups.

The present invention additionally provides the use of polyester polyols which are produced by the process described above for producing polyurethane. Polyurethane is a versatile material which is used in many areas. Owing to the wide variety of raw materials that can be used, products with extremely varied properties can be produced, for example rigid foams for insulation, flexible block foams for mattresses, moulded flexible foams for car seats and seat cushions, acoustic foams for sound insulation, thermoplastic foams, shoe foams or microcellular foams, but also compact casting systems and thermoplastic polyurethanes.

The invention is explained below with the aid of examples.

### EXAMPLES

List of the Raw Materials Used in the Examples

Terephthalic acid Interquisa

Phthalic anhydride (PA): Industrial PA from Lanxcss

Diethylene glycol (DEG): DEG from Ineos

Tin(II) chloride dihydrate from Aldrich

Polyethylene glycol (PEG 200) PEG 200 from Ineos.

Equipment and Analytical Methods Used:

Viscometer: MCR 51 from Anton Paar

Hydroxyl number: based on standard DIN 53240

Acid number: based on standard DIN 53402

Production of Polyester Polyols

### Example A-1

### According to the Invention

In a 4-litre, 4-neck flask equipped with a Pilz heating mantle, mechanical stirrer, internal thermometer, 40 cm packed column, column head, descending high-efficiency condenser and dry ice cooled receiver, as well as a membrane vacuum pump, under nitrogen blanketing at 140° C., 2134 g (10.67 mol) PEG 200 were initially charged, 1106 g (6.66 mol) terephthalic acid were stirred in and 78 mg tin(II) chloride dihydrate were added. The reaction temperature was increased to 230° C. and the mixture was stirred under standard pressure for 22 hours, during which dioxane-free water of reaction separated out. A further 78 mg tin(II) chloride dihydrate were added, the pressure was reduced over the course of 3 hours to 30 mbar and the reaction was completed for a further 17 hours. The acid number and hydroxyl number were determined as 0.2 and 130 mg KOH/g respectively and 403 (3.8 mol) diethylene glycol were then added and the mixture was stirred for 5 hours at 200° C. at standard pressure under nitrogen blanketing, during which no more water of reaction was separated out.

Analysis of the Polyester Polyol:

Hydroxyl number: 243.8 mg KOH/g

Acid number: 0.2 mg KOH/g

Viscosity: 1210 mPas (25° C.)

The quantity of 1,4-dioxane formed in the water of reaction was determined by gas chromatography as 0 wt. % and in the resulting polyester as 0.1 wt. %.

### Example A-2

### According to the Invention

Method as described in A-1; quantities used, see Table 1.

### Example A-3

### According to the Invention

In a reaction apparatus as in Ex. A-1, under nitrogen blanketing at 140° C., 2134 g (10.67 mol) PEG 200 were initially charged, 553 g (3.33 mol) terephthalic acid stirred in and 78 mg tin(II) chloride dihydrate added. The reaction temperature was increased to 230° C. and the mixture was stirred at standard pressure for 4 hours, during which dioxane-free water of reaction separated out. The temperature was reduced to 180° C., 493 g (3.33 mol) PA were added and the reaction was continued at standard pressure for a further 3 hours. A further 78 mg tin(II) chloride dihydrate were added, the pressure was reduced over the course of 3 hours to 30 mbar and the reaction was completed for a further 15 hours. The acid number and hydroxyl number were determined as 0.9 and 133.1 mg KOH/g respectively and 395 (3.73 mol) diethylene glycol were then added and the mixture was stirred for 5 hours at 200° C. at standard pressure under nitrogen blanketing, during which no more water of reaction was separated out.

Analysis of the Polyester Polyol:

Hydroxyl number: 242.9 mg KOH/g

Acid number: 0.7 mg KOH/g

Viscosity: 1300 mPas (25° C.)

The quantity of 1,4-dioxane formed in the water of reaction was determined by gas chromatography as 0 wt. % and in the resulting polyester as 0.1 wt. %.

### Example A-4

### According to the Invention

Method as described in A-3; quantities used, see Table 1.

TABLE 1
Examples A-1 to A-4. 50 ppm tin(II) chloride dihydrate were used
as catalyst in each case.

| | | Example | | | |
|---|---|---|---|---|---|
| | | A-1 | A-2 | A-3 | A-4 |
| Step 1 | | | | | |
| PEG 200 | [mol] | 10.67 | 10.67 | 10.67 | 10.67 |
| | [g] | 2134 | 2134 | 2134 | 2134 |
| Terephthalic acid | [mol] | 6.66 | 6.66 | 3.33 | 3.33 |
| | [g] | 1106 | 1106 | 553 | 553 |
| Step 2 | | | | | |
| Phthalic anhydride | [mol] | | | 3.33 | 3.33 |
| | [g] | | | 493 | 493 |
| Step 3 | | | | | |
| Diethylene | [mol] | 3.80 | 2.39 | 3.73 | 2.20 |
| glycol | [g] | 403 | 253 | 395 | 233 |
| Hydroxyl number | [mg KOH/g] | 243.8 | 205.7 | 242.9 | 202.5 |
| Acid number | [mg KOH/g] | 0.2 | 0.2 | 0.7 | 0.6 |
| Max. reaction | [° C.] | 230 | 230 | 230 | 230 |
| temp. | | | | | |
| Running time | [h] | 48 | 48 | 30 | 30 |
| Dioxane, exp. | [g] | 0.34 | 0.34 | 0.34 | 0.32 |
| Mass of ester, | [g] | 3403 | 3253 | 3395 | 3232 |
| theor. | | | | | |
| Mass of ester, | [g] | 3402.7 | 3252.7 | 3394.7 | 3231.7 |
| without dioxane | | | | | |
| Dioxane/kg ester | [g dioxane/ | 0.1 | 0.1 | 0.1 | 0.1 |
| | kg ester] | | | | |
| Dioxane/kg | [g dioxane/ | 0.84 | 1.34 | 0.86 | 1.37 |
| diethylene glycol | kg | | | | |
| | diethylene | | | | |
| | glycol] | | | | |
| Viscosity (at | [mPas] | 1210 | 1850 | 1300 | 1850 |
| 25° C.) | | | | | |

### Example A-5

### (C) (Comparison)

In an apparatus as in Example A-1, under nitrogen blanketing at 140° C., 441 g (2.98 mol) PA were initially charged and 285 g (2.69 mol) diethylene glycol added slowly and the mixture was stirred for one hour. Then, 2087 g (10.44 mol) PEG 200 and 495 g (2.98 mol) terephthalic acid were stirred in, together with 78 mg tin (II) chloride dihydrate, the temperature was increased to 230° C. and water of reaction was distilled off at standard pressure over 5 hours. Then, a further 78 mg tin (II) chloride dihydrate were added, the pressure was reduced slowly to a final value of 80 mbar and the reaction was completed under these conditions for a further 15 hours.

Throughout the entire reaction, distillates were collected in a receiver cooled with dry ice. The quantity of 1,4-dioxane formed was determined by gas chromatography as 12.8 g.

Analysis of the Polyester Polyol

Hydroxyl number: 232.7 mg KOH/g

Acid number: 0.2 mg KOH/g

Viscosity: 1160 mPas (25° C.)

Quantity of polyester polyol formed: 3135.2 g

Quantity of dioxane based on quantity of diethylene glycol used:

12.8 g/0.285 kg=44.9 g dioxane/kg diethylene glycol

### Example A-6

### (C) (Comparison)

Method as described in A-5 (C); quantities used, see Table 2.

TABLE 2
Examples A-5(C) and A6(C).
50 ppm tin(II) chloride dihydrate were used as catalyst in each
case.

Example

| | | A-5(C) | A-6(C) |
|---|---|---|---|
| Step 1 | | | |
| Phthalic anhydride | [mol] | 2.98 | 3.26 |
| | [g] | 441 | 542 |
| Diethylene glycol | [mol] | 2.69 | 2.94 |
| | [g] | 285 | 311 |
| Step 2 | | | |
| PEG 200 | [mol] | 10.44 | 9.20 |
| | [g] | 2087 | 1841 |
| Terephthalic acid | [mol] | 2.98 | 3.26 |
| | [g] | 495 | 542 |
| Hydroxyl number | [mg KOH/g] | 232.5 | 200.5 |
| Acid number | [mg KOH/g] | 0.2 | 0.7 |
| Max. reaction temp. | [° C.] | 230 | 230 |
| Running time | [h] | 21 | 36 |
| Dioxane, exp. | [g] | 12.8 | 12.4 |
| Mass of ester, theor. | [g] | 3148 | 3004 |
| Mass of ester, | [g] | 3135.2 | 2991.6 |
| without dioxane | | | |
| Dioxane/kg ester | [g dioxane/kg ester] | 4.08 | 4.15 |
| Dioxane/kg | [g dioxane/kg | 44.9 | 39.9 |
| diethylene glycol | diethylene glycol] | | |
| Viscosity (at 25° C.) | [mPas] | 1160 | 1870 |

A comparison of Tables 1 and 2 shows that the proportion of the diethylene glycol that is converted to dioxane depends strongly on the method selected with comparable formulations. Processes that are not according to the invention give 44.9 and 39.9 g dioxane respectively per kg of DEG used, whereas variants according to the invention have values of 0.84 to 1.37 in this respect.

Raw Materials for Rigid Foams:

a.) Polyesters from Ex. A-1, A-2, A-3, A-4, A-5(C) and A-6(C)

Foam additive, consisting of b.)-f.):

b.) TCPP, tris(1-chloro-2-propyl)phosphate from Lanxess

c.) TEP, triethyl phosphate from Lanxess

d.) Additive 1132 from Bayer MaterialScience

e.) PET V 657, trifunctional polyether polyol with a molecular weight of approx. 660 Da. from Bayer MaterialScience AG

f.) Polyether-polysiloxane copolymer stabiliser from Evonik

g.) Activator: Desmorapid VP.PU 30HB13 A from BMS

h.) Desmodur VP.PU 44V70L, polyisocyanate from Bayer MaterialScience

i.) n-pentane, Kremer&Martin

On a laboratory scale, all the raw materials for the rigid foam formulation, with the exception of the polyisocyanate component, were weighed into a paper cup, temperature-controlled at 23° C., mixed using a Pendraulik laboratory mixer (e.g. Type LM-34 from Pendraulik) and volatilised blowing agent (pentane) was optionally added. Next, with stirring, the polyisocyanate component (also temperature-controlled at 23° C.) was added to the polyol mixture, this was mixed intensively and the reaction mixture was poured into moulds. To determine adhesion and defects, moulds are used which are provided with a metallic covering layer (Corus). The foam hardness is determined by determining the depth of penetration in mm after 2.5 minutes using a defined weight. The reaction was allowed to continue for at least a further 24 hours at 23° C. and then the following properties were determined:
Fire: BVD test in accordance with the Swiss Basic Test to Determine the Combustibility of Building Materials from the Vereinigung kantonaler Feuerversicherungen [Association of Cantonal Fire Insurers] in the edition of 1988, with the supplements of 1990, 1994, 1995 and 2005 (obtainable from Vereinigung kantonaler Feuerversicherungen, Bundesstr. 20, 3011 Bern, Switzerland).
Adhesion: Determined by peeling off the foamed covering layer and establishing the force needed for this purpose using a spring balance
Defects: Visual evaluation for void formation. A distinction was made between “no, little, moderate and marked” void formation

TABLE 3
Formulations for rigid foams

| | | B-1 | B-2 | B-3 | B-4 | B-5(C) | B-6(C) |
|---|---|---|---|---|---|---|---|
| Polyester polyol | [pts.] | 63.8 | | | | | |
| from Ex. A-1 | | | | | | | |
| Polyester polyol | [pts.] | | 63.8 | | | | |
| from Ex. A-2 | | | | | | | |
| Polyester polyol | [pts.] | | | 63.8 | | | |
| from Ex. A-3 | | | | | | | |
| Polyester polyol | [pts.] | | | | 63.8 | | |
| from Ex. A-4 | | | | | | | |
| Polyester polyol | [pts.] | | | | | 63.8 | |
| from Ex. A-5(C) | | | | | | | |
| Polyester polyol | [pts.] | | | | | | 63.8 |
| from Ex. A-6(C) | | | | | | | |
| Foam additive | [pts.] | 36.2 | 36.2 | 36.2 | 36.2 | 36.2 | 36.2 |
| Pentane | [pts.] | 16.2 | 15 | 15.9 | 14.6 | 15.9 | 14.8 |
| Activator | [pts.] | 4.0 | 3.8 | 4.0 | 3.5 | 4.0 | 3.8 |
| Desmodur 44V70L | [pts.] | 169 | 148 | 165 | 143 | 163 | 146 |
| Rigid foam properties | | | | | | | |
| Fire class/ | Class | Cl. 5 | Cl. 5 | Cl. 5 | Cl. 5 | Cl. 5 | Cl. 5 |
| flame height | [mm] | 110-120 | 120 | 127 | 120 | 110-120 | 120-140 |
| Adhesion | [N] | 40 | 50 | 42 | 50 | 52 | 50 |
| Defects | | moderate | little | little | little | little | moderate |
| | | to marked | | | | | |
| Hardness | [mm] | 5 | 8 | 4 | 8 | 4 | 7 |

Table 3 shows that all the foams achieve fire class 5 and display an approximately equal level of properties.

However, while B-1 to B-4 are based on polyester polyols according to the invention, which were characterised in that, among other things, virtually no dioxane was formed as a by-product during their production, foams B-5(C) and B-6(C) are based on polyester polyols which fared dramatically worse in this respect. The foams according to the invention therefore have important advantages in an overall view of their production.

What is claimed is:

1. A one-step process for producing polyester polyols, comprising:
a) mixing
(A) at least one compound selected from the group consisting of aromatic dicarboxylic acids, alkyl esters of aromatic dicarboxylic acids, and anhydrides of aromatic dicarboxylic acids,
and
(B) at least one α,ω-diol,
thereby forming a polyester polyol, wherein the molar ratio of components (B) to (A) is in the range of 20:1 to 1.05:1 and the proportion by weight of components (A) and (B) in total, based on the total weight of the mixture obtained in step c), is in the range of between 55 and 99 wt. %, and
b) adding (C) phthalic anhydride to the polyester polyol obtained in step a), in a molar ratio of components (C) to (A) added in step a) in the range of 0:1 to 2:1, thereby forming a polyester polyol, and
c) adding (D) diethylene glycol to the polyester polyol obtained in step b), in a proportion by weight of 1 to 35 wt. %, based on the total weight of the mixture, thereby forming of a polyester polyol,
wherein the polyester polyol obtained in step b) has a higher molecular weight than the polyester polyol obtained in step c).

2. The process according to claim 1, wherein (A) said compound is an the aromatic dicarboxylic acid selected from the group consisting of phthalic acid, tercphthalic acid, isophthalic acid and mixtures thereof.

3. The process according to claim 1, wherein (B) said α,ω-diol is selected from the group consisting of ethylene glycol, triethylene glycol, tetraethylene glycol, pentaethylene glycol and higher homologs thereof with number-average molecular weights of 150 to 410 g/mol.

4. The process according to claim 1, wherein the molar ratio of components (B):(A) is in the range of 1.1:1.0 to 10.0:1.0.

5. The process according to claim 1, wherein the molecular weight of the polyester polyol obtained from step a) is in the range of between 4000 and 2000.

6. The process according to claim 1, wherein the OH number of the polyester polyol obtained from step a) is in the range of between 28 and 560 KOH/kg.

7. The process according to claim 1, wherein the molecular weight of the polyester polyol obtained from step b) is in the range of between 4500 and 600.

8. The process according to claim 1, wherein the OH number of the polyester polyol obtained from step b) is in the range of between 25 and 190 g KOH/kg.

9. The process according to claim 1, wherein the viscosity of the polyester polyol obtained from step b) at a temperature of 25° C. is in the range of between 400 and 20000 mPas.

10. The process according to claim 1, wherein the proportion by weight of components (A) and (B) in total, based on the total weight of the mixture, is in the range of between 60 and 90 wt. %.

11. A polyester polyol obtainable according to the process of claim 1.

12. A process for producing a polyurethane foam, which may optionally contain polyisocyanurate groups, which comprises reacting
a) a polyester polyol is produced by the process of claim 1,
with
b) at least one polyisocyanate-containing component, in the presence of
c) one or more blowing agents,
d) one or more catalysts,
and, optionally, in the presence of
d) one or more flame retardants and other auxiliary substances and additives, and, optionally, with
at least one compound having at least two isocyanate-reactive groups, which differs from said polyester polyol a).

13. A polyurethane loam, which may optionally contain polyisocyanurate groups, produced by the process according to claim 12.

14. (canceled)
